(19) United States
(12) Patent Application Publication
(10) Pub. No.: US 2006/0015013 A1
(43) Pub. Date: Jan. 19, 2006

(54) Device and method for in vivo illumination

(76) Inventors: Zvika Gilad, Haifa (IL); Semion Khait, Tiberias (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/879,258

(22) Filed: Jun. 30, 2004

(51) Int. Cl. A61B 1/06

(52) U.S. Cl. 600/160

(57) ABSTRACT

An in vivo sensing device including an illumination sub system. The illumination sub system includes, for example, a conductive ring and/or a conductive step for and an illumination sources positioned at a selected angle.

### FIELD OF THE INVENTION

The present invention relates to a device useful for in-vivo imaging, more specifically to a device for providing illumination in-vivo.

### BACKGROUND OF THE INVENTION

Devices helpful in providing in-vivo imaging are known in the field. Autonomous in-vivo imaging devices, such as swallowable capsules or other devices may move through a body lumen, imaging as they move along. In vivo imaging may require in-vivo illumination, for example, using one or more LEDs or other suitable illumination sources positioned inside an in-vivo imaging device. Typically, it is required that the illumination be directed outwards from the device, to the body lumen being imaged.

In some in vivo devices, such as ingestible imaging capsules, the electronic components within the capsule, such as illumination sources, may be arranged on a board or on several boards, such as on a printed circuit board (PCB). In some cases proper alignment or positioning of components, such as the illumination sources, may not be easily achieved.

### SUMMARY OF THE INVENTION

There is provided, in accordance with some embodiments of the present invention an in vivo imaging device having an illumination sub system. According to one embodiment the illumination sub system may include, for example, a base or support for holding one or more light sources, for example, LEDs or other suitable illumination sources.

According to one embodiment of the present invention the base may include a conductive ring and/or other components for holding illumination sources at a selected angle.

According to another embodiment of the present invention a support, for example a PCB, or a set of supports may form a structure on which illumination sources may be positioned. For example, a set of supports may be designed in the shape of a “top hat” or other suitable structure so as to enable an illumination source positioned on the structure to be facing, for example, outwards, at a selected angle.

In another embodiment the support may be manufactured according to several designs, enabling the support to fit into devices of different shapes.

### BRIEF DESCRIPTION OF THE DRAWINGS

The principles and operation of the system, apparatus, and method according to the present invention may be better understood with reference to the drawings, and the following description, it being understood that these drawings are given for illustrative purposes-only and are not meant to be limiting, wherein:

FIG. 1 shows a schematic illustration of an in-vivo imaging device, according to some embodiments of the present invention;

FIGS. 2A-2D are schematic diagrams of supports and constructions, according to some embodiments of the present invention;

FIG. 3 shows a schematic illustration of a flexible support, according to some embodiments of the present invention;

FIG. 4 is a flow chart of a method of illuminating a body lumen in accordance with an embodiment of the invention; and

FIG. 5 is a flow chart of a method of illuminating a body lumen in accordance with another embodiment of the invention.

It should be noted that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

Illumination sources used with embodiments of the present invention may include, for example, Light Emitting Diodes (LEDs), incandescent sources, or other suitable light sources that may enable in-vivo illumination, and may include devices providing electromagnetic radiation within the visible spectrum, outside of the visible spectrum, and further a combination of visible and non-visible electromagnetic radiation.

Embodiments of the invention may typically be autonomous and typically self-contained. For example, a device according to some embodiments may be a capsule or other unit where all the components are substantially contained within a container or shell, and where the device does not require wires or cables in order to receive power or transmit information, for example. The device may communicate with an external receiving and display system to provide display of data, control, or other functions. -Power may be provided, for example, by an internal battery or a wireless receiving system. Other embodiments may have other configurations and capabilities. Components in some cases may be distributed over multiple sites or units and Control information may be received from an external source.

Some embodiments of the present invention are directed to a typically swallowable in-vivo device that may be used for recording and transmitting in vivo data, such as, for example, from-the entire length of the gastrointestinal (GI) tract, to a receiving and/or processing unit. Other embodiments need not be swallowable or autonomous, and may have other shapes or configurations. According to some embodiments the in vivo device may include an image sensor, however, other sensors may be used. Devices according to embodiments of the present invention may be similar to embodiments described in International Application WO 01/65995 and/or in U.S. Pat. No. 5,604,531, each of which are assigned to the common assignee of the present invention and each of which are hereby incorporated by reference in their entirety. Furthermore, receiving, storage, processing and/or display systems suitable for use with embodiments of the present invention may be similar to embodiments described in WO 01/65995 and/or in U.S. Pat. No. 5,604,531. Of course, devices, systems, structures, functionalities and methods as described herein may have other configurations, sets of components and processes etc.

It should be noted that while a device, system and method in accordance with some embodiments of the invention may be used, for example, in a human body, the invention is not limited in this respect. For example, some embodiments of the invention may be used in conjunction or inserted into a non-human body, e.g., a dog, a cat, a rat, a cow, or other animals, pets, laboratory animals, etc.

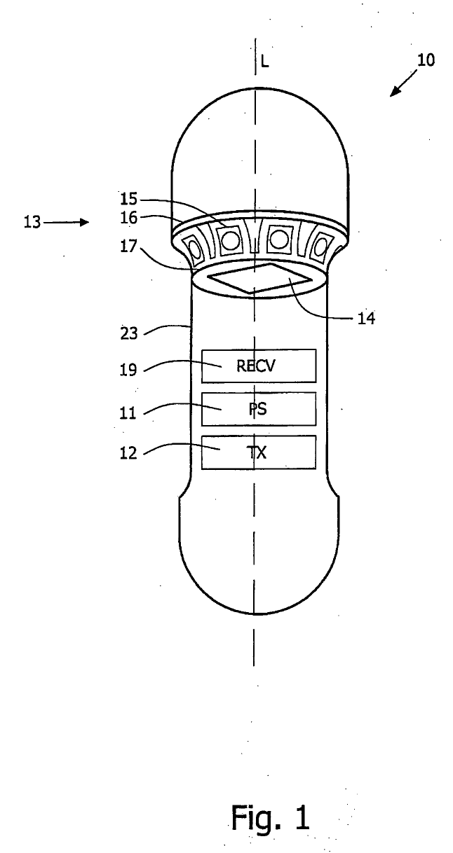

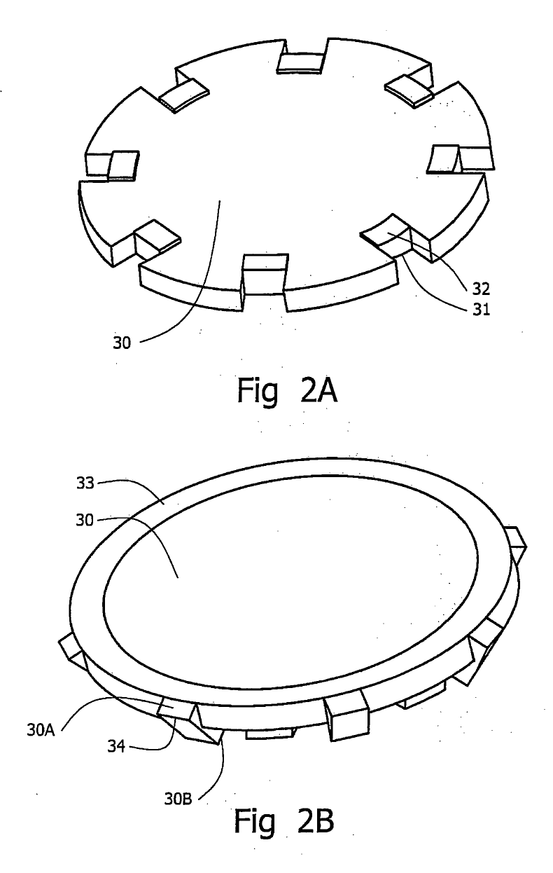

Reference is now made to FIG. 1, which is a schematic illustration of an in-vivo imaging device 10 with an illumination sub-system 13, according to some embodiments of the present invention. Device 10, which may be a swallowable capsule, may include, for example, a power source 11, a transmitter 12, an imager 14 and possibly a receiver 19. Illumination sub-system 13 may include for example a base 17, for example, a printed circuit board (PCB) board or other suitable support, including one or more illumination sources 15, such as LEDs or other suitable illumination sources. Base 17 may include one or more components, for example, conductive rings, and/or conductive step 16. Base 17 may include illumination sources 15 positioned at a selected angle relative to the longitudinal axis (L) of the device 10. Typically, the imager 14 faces generally in the direction of axis L. Thus, the direction of imaging, which may be the direction in which the imager is facing, may coincide with an axis (e.g., axis L) of the device 10. However, other arrangements are possible; for example the illumination sources need not be angled relative to a specific axis and need not be angled relative to a viewing or imaging direction. Illumination source 15 may be positioned, for example on base 17 and/or on a conductive ring or conductive step 16 and/or stepped substrate (as will be described, for example, with reference to FIG. 2D). A stepped substrate may be, for example, a stepped PCB, e.g., a substrate such as a PCB or a set of substrates designed in the shape of a “top hat” or other suitable structure so as to enable an illumination source such as an LED positioned on the structure to be facing outwards at a selected angle. Other designs, components, elements etc. may be used. Other arrangement directions may be chosen, for example, to create different angles of illumination source 15 in order to illuminate, for example, a selected field of view. Other structures may be used in addition to and/or in place of rings, steps, etc.

Device 10 as depicted in FIG. 1 and according to one embodiment is generally capsule shaped, and may be easily swallowed and passively passed through the entire GI tract, pushed along, for example, by natural peristalsis. Nonetheless, it should be noted that device 10 may be of any shape and size suitable for being inserted into and passing through a body lumen or cavity, such as spherical, oval, cylindrical, etc. or other suitable shapes. Furthermore, device 10 or various embodiments that may include at least some components of device 10 may be attached or affixed on to an instrument that is inserted into body lumens and cavities, such as, for example, on an endoscope, laparoscope, needle, catheter etc.

According to one embodiment, device 10 includes a convex window 23. According to some embodiments one or more illumination source(s) 15 may be arranged in a ring and may be placed in close proximity to the convex window 23. Typically the structure formed by subsystem 13 according to embodiments of the invention, enables the illumination source(s) 15 to be positioned in proximity to a curved window, such as a convex window, and to conform to the shape of the window and/or device, so as to avoid phenomena (such as backscatter) usually associated with illuminating from within a window.

According to an embodiment of the present invention, as can be seen with reference to FIG. 2A, grooves or indentations 31 may be formed or cut out of a PCB or other suitable support 30, for holding one or more illumination sources 15, for example, LEDs. For example, angled channels or cutouts in one or more PCBs may be used to hold one or more illumination sources. Conductive pads 32, for example, metal pads, may be placed or molded in grooves 31, to provide connections for illumination sources. In FIG. 2B, which is a schematic bottom view, a conductive ring 33 may be connected to pads 32, for example, on the bottom of the PCB or other support 30, to provide conductivity between all the pads, and to provide a base for positioning the illumination sources. Illumination sources 34 may be placed on support 30 with one end 30A being in contact with conductive ring 33, and another end 30B being in contact with the backs of grooves 31, thereby facing an angle determined by the conductive ring and the backs of the grooves. Any suitable angle may be provided for the placement of the illumination sources.

FIG. 2C, which is a schematic view from the top, illustrates the addition of resistors 35 adjacent to illumination sources 34, according to one embodiment of the invention.

According to another embodiment of the present invention, as can be seen, for example, with reference to FIG. 2D, a PCB or other substrate with surfaces on two or more planes may be provided. An outer ring 36 in a first plane may be connected to an inner ring 37 which may be of smaller diameter and on a parallel plane to the first plane, for example, being higher and narrower than outer ring 36. Conductive pads 32 may be placed on both outer ring 36 and inner ring 37, enabling illumination sources 34 to be placed at an angle, leaning on both the outer ring 36 and inner ring 37. According to one embodiment of the present invention, any suitable angle may be provided for the placement of the illumination sources. According to another embodiment of the present invention, any suitable number of PCB planes may be used.

According to one embodiment of the present invention, a support including ceramic may be used as a base on which to place illumination sources. Ceramic may be provided with grooves and pads for the placement of light sources. In one embodiment a ceramic cone may be provided, such that light sources placed therein may transmit light at an angle created by the slope of the cone shaped ceramic.

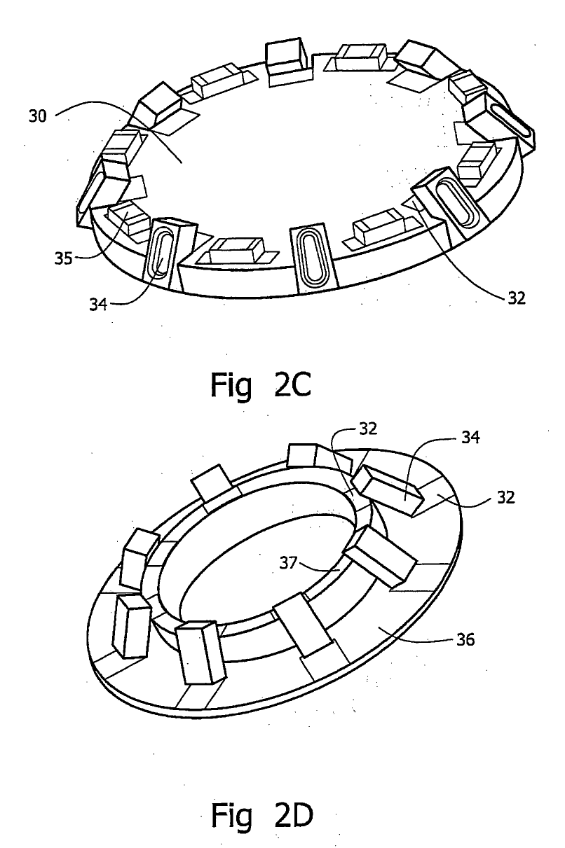

Reference is now made to FIG. 3 showing an exemplary embodiment of a flexible circuit board 50 shape after it has been folded and inserted into an in vivo device, for example, a capsule. According to one embodiment of the present invention, flexible circuit board portions 54 and 56 may be folded upon insertion so that they are “C” shaped, facing each other. According to other embodiments portions 54 and 56 need not be flexible. According to one embodiment, flexible leaves 58 include illumination sources 15 and may, for example, bend in a range of degrees upon inserting the circuit board into a capsule housing tube so as to enable, for example, an outwards illumination at different angles. The illumination angle may be determined by the housing tube shape. For example, flexible leaves 58 may enable to position illumination source(s) 15 in proximity to a curved window (e.g., a convex window) within a device.

According to some embodiments circuit board 50 may include other components of the device such as, imager 14 and antenna 53, typically associated with a transmitter for transmitting images from imager 14. Circuit board 50 may further include contact points 52 to connect additional components, such as a power source.

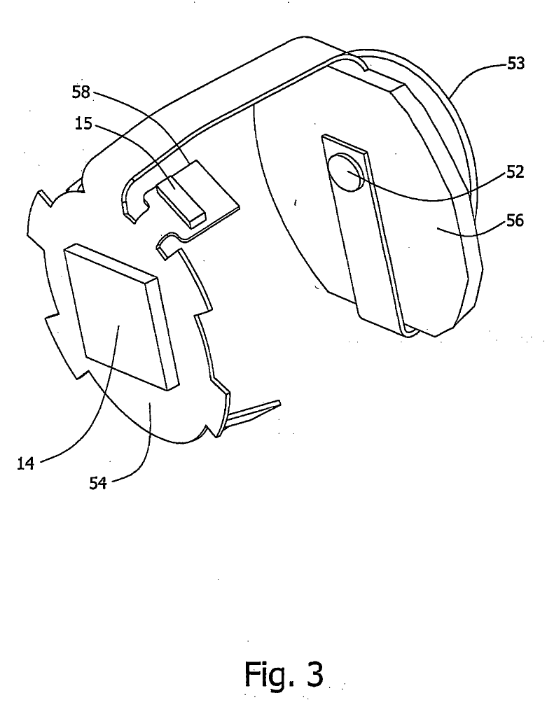

According to some embodiments of the present invention, as shown, for example, in FIG. 4, a method is provided for in-vivo illumination that may include, providing, in an in vivo imaging device, an illumination source at an angle (410). According to some embodiments the angle may be relative to the direction of imaging. According to other embodiments the angle may be relative to an axis of the device. Another step includes illuminating a body lumen (420). According to one embodiment the angle is typically less than 90 degrees, to the direction of imaging, which may coincide, for example, with an axis of the imaging device. Embodiments of the invention may achieve a broad field-of-view, by using, for example, a panoramic imaging device which includes a reflective element, for example, a curved or other suitably shaped mirror, to capture a panoramic image. According to one embodiment a segment of the outside wall of the panoramic imaging device may be partially or entirely transparent. The illumination in a panoramic imaging device may be provided by angled illuminations sources, according to an embodiment of the invention. An in-vivo lumen may be illuminated using the light source, which may be enabled to provide light at a wide angled. However, the method may be implemented using other in-vivo devices having other suitable structures. Any combination of the above steps may be implemented. Further, other steps or series of steps may be used.

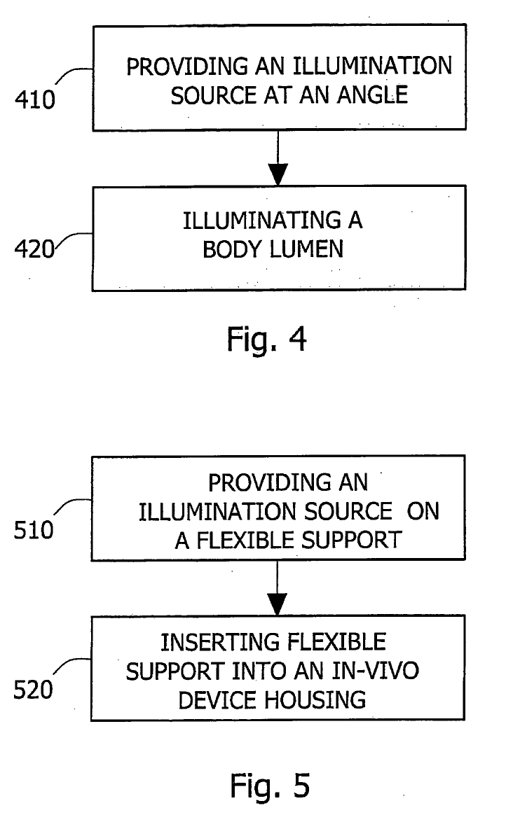

A method for providing in vivo illumination at an angle according to another embodiment is shown in FIG. 5. According to one embodiment the method may include providing an illumination source on a flexible support (510) and inserting the support into a housing of an in vivo device (520). Typically, the support will assume the shape of the device housing. According to one embodiment an illumination source provided on a flexible support may be, upon insertion into a device housing, placed against a transparent section of the housing (e.g., an optical window), typically at an angle that is dictated by the shape of the housing, thus enabling illumination of an area out side of the device, for any shape of device. According to one embodiment the support may include additional components of the device.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. An in vivo imaging device having a direction of imaging, said device comprising an illumination source positioned at an angle, of 0-90 degrees relative to the direction of imaging.

2. The device according to claim 1 comprising a support for holding the illumination source.

3. The device according to claim 2 wherein the support is flexible.

4. The device according to claim 2 wherein the support is a PCB.

5. The device according to claim 2, wherein on the support is a stepped support.

6. The device according to claim 1, wherein the illumination source is positioned on a conductive ring.

7. The device according to claim 1, wherein the illumination source is in contact with a conductive ring and a stepped support.

8. The device according to claim 1, wherein said imaging device is an autonomous in-vivo imaging device.

9. The device according to claim 1 comprising an imager.

10. The device according to claim 1 comprising a transmitter.

11. The device according to claim 1 comprising at least one component selected from the group consisting of a power source, a sensor, a receiver, a processor, or a combination thereof.

12. The device according to claim 1 comprising a convex window.

13. The device according to claim 12 wherein the illumination source is in close proximity to the convex window.

14. A method for in vivo imaging comprising
providing an in vivo imaging device having a direction of imaging and comprising an illumination source at an angle, of between 0-90 degrees to the direction of imaging;
illuminating a body lumen; and
obtaining images if the body lumen.

15. The method according to claim 14 comprising positioning said illumination source on a conductive ring.

16. The method according to claim 14 comprising positioning said illumination source on a stepped support.

17. The method according to claim 14 comprising positioning said illumination source on a flexible support.

18. The method according to claim 14 comprising positioning said illumination source on a two plane support.

19. An in vivo imaging device comprising a convex window.

20. The device according to claim 19 comprising an imager facing in a direction of imaging and an illumination, the illumination source positioned at an angle, of 0-90 degrees relative to the direction of imaging.

21. The device according to claim 20 comprising a support for holding the illumination source.

22. The device according to claim 21 wherein the support is flexible.

23. The device according to claim 21 wherein the support is a PCB.

24. The device according to claim 21, wherein on the support is a stepped support.
